(19) United States
(12) Patent Application Publication
(10) Pub. No.: US 2026/0263696 A1
(43) Pub. Date: Sep. 10, 2026

(54) DEVICE FOR ADMINISTERING A FLUID SUBSTANCE

(71) Applicant: Ypsomed AG, Burgdorf (CH)

(72) Inventors: Oliver Glauser, Bern (CH); Dominik Zumstein, Lyss (CH); Simon Martin Bosshard, Hindelbank (CH); Christian Schrul, Oberburg (CH); Markus Tschirren, Burgdorf (CH); Peter Loser, Herrenschwanden (CH)

(21) Appl. No.: 19/661,276

(22) Filed: Apr. 28, 2026

Related U.S. Application Data
(63) Continuation of application No. PCT/EP2024/080540, filed on Oct. 29, 2024.

(30) Foreign Application Priority Data
Oct. 31, 2023 (EP) 23207090.4

(51) Int. Cl. A61M 5/20; A61M 5/315; A61M 5/32

(52) U.S. Cl. CPC A61M 5/2033; A61M 5/3157; A61M 2005/3267

(57) ABSTRACT

A device for administering or discharging a fluid substance includes a housing and a holding element, which is fixed with respect to the housing and in which an injection spring is received. The holding element can be moved radially outwards or radially deflected between an engagement position, in which the holding element is engaged with the injection spring, and a release position, in which the holding element is disengaged from the injection spring. During an axial movement of a needle guard element from the distal position to the proximal position, the holding element can disengage from the injection spring to administer or discharge the substance from the product container through the needle.

### CROSS-REFERENCE TO RELATED APPLICATIONS

This application is a continuation of International Patent Application No. PCT/EP2024/080540, filed Oct. 29, 2024, entitled “DEVICE FOR ADMINISTERING A FLUID SUBSTANCE,” which in turn claims priority to European Patent Application No. 23207090.4, filed Oct. 31, 2023, entitled “DEVICE FOR ADMINISTERING A FLUID SUBSTANCE”, each of which is incorporated by reference herein, in the entirety and for all purposes.

### TECHNICAL FIELD

The disclosure relates to a device with a longitudinal axis (L) for administering or discharging a fluid substance, in particular a medicament.

### BACKGROUND

Devices for administering or discharging a fluid substance are known from the prior art. From EP 2742962 B1, for example, an autoinjector with an axially displaceable holding element is known, wherein the holding element can disengage from a piston rod. Furthermore, the piston rod, which is held by an injection spring, can be moved in the distal direction to act on a stopper of a syringe, so that a fluid substance is administered or discharged.

A disadvantage of this autoinjector is that is has a complex design and a large number of components.

### SUMMARY

It is an object of the present disclosure to provide a device for administering or discharging a fluid substance which device is simple in design and has few components.

In the following, the proximal direction of a device for administering or discharging a fluid substance means the direction towards the housing-side end, and the distal direction of this device means the direction towards the needle-side or cannula-side end.

The device according to the present disclosure, includes a longitudinal axis (L) for administering or discharging a fluid substance, in particular a medicament, and has a housing. The housing may be elongated. The housing may be sleeve-shaped and/or cylindrical, in particular circular-cylindrical. A product container is accommodated in the housing in axially fixed fashion. The product container contains the substance to be administered. Furthermore, a stopper is provided in the product container, which is accommodated in the product container in an axially displaceable fashion. In addition, the product container has a needle or cannula through which the substance can be administered or discharged. The stopper may be arranged to be axially displaceable from a proximal end of the product container to a distal end of the product container in order to administer or discharge the substance provided in the product container. The product container may be a syringe comprising a syringe body and a needle or cannula that is fixedly or non-detachably received on the distal end of the syringe body. Alternatively, the product container can be designed as a cartridge with a cartridge body and an attachable and removable needle unit.

Furthermore, the device according to the present disclosure may include a needle guard element for the needle or cannula that is movable relative to the housing. The needle guard element can be sleeve-shaped and/or cylindrical, in particular circular-cylindrical. The needle guard can be used to protect against puncture wounds from the needle or cannula, in particular from a distal tip of the needle or a distal tip of the cannula. Furthermore, the device has a needle guard spring, which is coupled to the needle guard element, where the needle guard element is axially movable from a distal position in which the distal tip of the needle or cannula is surrounded by the needle guard element and to a proximal position in which the distal tip of the needle or cannula is released from the needle guard element.

Furthermore, the device according to the present disclosure has an injection spring, which provides the energy to effect the administration of the substance and which cooperates with the stopper to administer or discharge the substance from the product container through the needle or through the cannula. The device can be configured as an autoinjector. Autoinjectors are often described as devices that, after activation, automatically dispense a substance to be administered to a patient.

The needle guard spring and the injection spring may be configured as compression springs. The two springs may be particularly configured as helical springs, in particular as helical springs with one or more coils. However, other springs can also be provided. The needle guard spring and the injection spring may be pre-tensioned in the device according to the present disclosure. The needle guard spring and the injection spring may be configured to be tensionable and relaxable. The force of the needle guard spring may cause the needle guard element to be moved from the distal position to the proximal position and back to the distal position, or to another distal position different from the distal position. The force of the injection spring causes the injection spring to discharge or be able to discharge the entire amount of the substance to be administered or a portion thereof.

The device, according to the present disclosure, may also include a holding element arranged axially fixed relative to the housing, and in which the injection spring is accommodated, in particular so as to be pre-tensioned and relaxable. The holding element may be radially movable outwardly or radially deflectable between an engagement position, in which the holding element is engaged with the injection spring, and a release position, in which the holding element is disengaged from the injection spring. The holding element may be elongated and may extend along the longitudinal axis (L). The holding element may particularly include a holding arm and/or a holding cam radially movable outwards or radially deflectable. A plurality of holding arms and/or holding cams may be provided. The holding arm of the holding element may extend from a proximal end to a distal end, where the proximal end of the holding arm is arranged axially fixed to the housing or on an element axially fixed to the housing. The holding element, in particular the holding arm and/or the holding cam of the holding element, may be designed to be elastic. The holding element, in particular the holding arm and/or the holding cam of the holding element, may be designed to be elastic such that it is radially movable outwards or radially deflectable between an engagement position, in which it is in engagement with the injection spring, such as the coil or coils of the injection spring, and a release position in which it is disengaged from the injection spring, such as from the coil or coils of the injection spring. The holding element, in particular the holding arm of the holding element and/or the holding cam of the holding element, may be configured in such a way that the injection spring, in particular the coil or coils of the injection spring, can be held in engagement under pre-tension.

Furthermore, during the axial movement of the needle guard element from the distal position to the proximal position, the holding element, in particular the holding arm of the holding element, and more particularly the holding cam of the holding arm of the holding element, disengages from the injection spring in order to administer or discharge the substance from the product container through the needle or cannula. The needle guard element may be configured such that, during the axial movement of the needle guard element from the distal position to the proximal position, the holding element, in particular the holding arm of the holding element, and more particularly the holding cam of the holding arm of the holding element, disengages from the injection spring in order to administer or discharge the substance from the product container through the needle or cannula. The injection spring pre-tensioned in the holding element can cause a radial outward movement or a radial deflection of the holding element, in particular of the holding arm and/or the holding cam of the holding element. Furthermore, the injection spring can relax when the holding element, holding arm and/or holding cam is disengaged from the injection spring, thereby allowing the substance to be administered or discharged from the product container through the needle or cannula. The injection spring relaxes directly into the product container during administration or discharge of the fluid substance. The advantage of the device according to the present disclosure is its compact size, in particular the external dimensions of the device. The device according to the present disclosure is optimized in such a way that it has few components. Furthermore, the device according to the present disclosure is simple and robust in design.

In other embodiments, the holding element may have a different shape, where the holding element is configured such that it is axially fixed relative to the housing of the device and is radially movable outwardly or radially deflectable between the engagement position in which the holding element is engaged with the injection spring and a release position in which the holding element is disengaged from the injection spring. In the engagement position, the holding element can hold the injection spring in a pre-tensioned engagement. Furthermore, in the release position, the injection spring can relax, allowing the substance to be administered or discharged from the product container.

In order to administer or discharge the substance from the product container, in one embodiment of the device according to the present disclosure, the injection spring can act directly on the stopper in the product container. The injection spring relaxes directly into the product container during the administration or discharge of the fluid substance. This embodiment of the device according to the present disclosure has the advantage that the device thereby has a high administering or discharging capacity and a compact design. The injection spring can comprise a distal part and a proximal part. The distal part of the injection spring can be configured in such a way that the distal part of the injection spring acts as a damping element. When the injection spring impacts the stopper, the impact energy can be at least partially absorbed to prevent glass breakage. In the as-delivered state, the distal part of the injection spring can be relaxed, and only becomes loaded or tensioned upon impact with the stopper. The proximal part of the injection spring can serve to store the discharge energy or the administration energy. In its as-delivered state, the proximal part of the injection spring can be pre-tensioned or held in pre-tensioned engagement by the holding element. The axial engagement position of the holding element relative to the injection spring can thus divide the injection spring into the distal part and the proximal part. The distal and proximal parts of the injection spring can be made of different materials or have different structures or tensions.

In an alternative embodiment of the device according to the present disclosure, an adapter is provided between the injection spring and the stopper in order to administer or discharge the substance. In the engagement position, the holding element can hold the injection spring in a pre-tensioned engagement, while in the release position the holding element is disengaged from the injection spring. The injection spring thus acts on the stopper via the adapter in order to administer or discharge the substance. This embodiment of the device according to the present disclosure has the advantage that differently shaped injection springs and differently shaped stoppers can thereby be assembled and yet a device with a high administration rate and a compact design can be provided. The adapter can be elongated and can extend along the longitudinal axis (L). The adapter can be sleeve-shaped or made of solid material. In one embodiment of the device according to the present disclosure, the distal and proximal ends of the adapter can be designed such that the ends can come into stop contact with the stopper of the product container at one end and with the injection spring at the other. The adapter can also be designed as a damping element to reduce or correspondingly adjust the force of the injection spring. The adapter can also have a guide for guiding the injection spring. The guide for the adapter can be provided on an outer surface of the adapter. The guide extends parallel to the spring axis of the injection spring. This can prevent the injection spring from bending. In one embodiment of the device according to the present disclosure, the distal end of the injection spring can be supported on a part, in particular on a proximal part or middle part of the adapter, and the proximal end of the injection spring can be supported on a proximal part of the housing or on a proximal part of the holding element. In the device according to the present disclosure, the injection spring relaxes directly into the product container during the administration or discharge of the fluid substance. This allows the design of the device according to the present disclosure to be advantageously optimized. During this relaxation, the injection spring, and in particular the energy released by the injection spring, can act via the adapter on the stopper in order to administer or discharge the substance from the product container.

In another embodiment of the device, an adapter is provided between the injection spring and the stopper in order to administer or discharge the substance. In this embodiment of the device, the holding element can additionally or exclusively engage with or disengage from the adapter. The holding element, in particular a holding arm of the holding element or a holding cam of the holding element, is configured in such a way that the adapter can be held in engagement, so that the injection spring, in particular the coil or coils of the injection spring, are pre-tensioned.

Furthermore, during the axial movement of the needle guard element from the distal position to the proximal position, the holding element, in particular the holding arm of the holding element, and more particularly the holding cam of the holding arm or of the holding element, disengages from the adapter in order to administer or discharge the substance from the product container through the needle or cannula. The needle guard element can be designed such that, during the axial movement of the needle guard element from the distal position to the proximal position, the holding element, in particular the holding arm of the holding element, and more particularly the holding cam of the holding arm or of the holding element, disengages from the adapter in order to administer or discharge the substance from the product container through the needle or cannula. The injection spring pre-tensioned in the holding element can cause a radial outward movement or a radial deflection of the holding element, in particular of the holding arm of the holding element, particularly preferably of the holding cam of the holding arm or of the holding element. The injection spring can further relax when the holding element, in particular the holding arm of the holding element, and more particularly the holding cam of the holding arm or the holding element, is disengaged from the adapter, thereby allowing the substance to be administered or discharged from the product container through the needle or cannula. During this relaxation, the injection spring, and in particular the energy released by the injection spring, can act via the adapter on the stopper in order to administer or discharge the substance from the product container. The injection spring acts indirectly on the stopper in order to administer or discharge the substance from the product container. The injection spring relaxes directly into the product container during the administration or discharge of the fluid substance.

The advantage of the device according to the present disclosure is its compact size, in particular the external dimensions of the device. The device according to the present disclosure is optimized in such a way that it has few components. Furthermore, the device according to the present disclosure is simple and robust in design.

Furthermore, during administration or discharge of the substance, the injection spring, in particular the coils of the injection spring, can be relaxed relative to the holding element and over the holding element in order to generate an acoustic, visual and/or tactile discharge signal. The discharge signal can be generated by a relative movement between the holding element, in particular the holding arm of the holding element and/or the holding cam of the holding element, and the injection spring, in particular the coils of the injection spring. In particular, the coils of the injection spring, which relax during the discharging process, can slide over the holding element and/or over the holding arm of the holding element and/or the holding cam of the holding arm or the holding element in order to generate the discharge signal. The acoustic signal can be for example an audible rattling or clicking sound caused by a relative movement between the holding element, in particular the holding arm of the holding element and/or the holding cam of the holding arm or the holding element, and the coils of the injection spring during the administration or discharge of the substance. The holding element, the holding arm, or the holding cam can be designed as a click element. The visual signal may be seen, for example via a window, which can be provided in the housing of the device according to the present disclosure, through which the relaxation of the coils of the injection spring can be seen. The tactile signal can, for example, be a perceptible vibration when the coils of the injection spring relax relative to the holding element, in particular the holding arm of the holding element and/or the holding cam of the holding arm or of the holding element.

In a further embodiment of the device according to the present disclosure, the discharge signal can be generated by an additional holding element, in particular an additional holding arm of the holding element or an additional holding cam of the holding arm or of the holding element. The device according to the present disclosure can thus have a first holding element and a second holding element, in particular a first holding arm and a second holding arm and a first holding cam and a second holding cam. The first holding element, in particular the first holding arm or the first holding cam, can serve to hold the injection spring in or out of engagement, and the second holding element, in particular the second holding arm or the second holding cam, can serve to generate the discharge signal. The second holding element, the second holding arm or the second holding cam can be designed as a click element. The second holding element, in particular the second holding arm or the second holding cam, can be designed to be elastic. The second holding element, in particular the second holding arm or the second holding cam, can be elastically attached to the first holding element or to an element axially fixed to the housing.

The holding element or the holding arm of the holding element or the holding cam of the holding arm or the holding element for generating the discharge signal may be configured such that the frequency of the perceptible discharge signal decreases during the administration or discharging process in order to indicate to the patient that the administration or discharge is coming to an end. For example, the frequency of the audible rattling or clicking during the administration or release of the substance can decrease. Furthermore, the visible distance between the coils of the injection spring may increase during the administration or discharge of the substance. Furthermore, the frequency of the perceptible vibration during the administration or discharge of the substance can for example decrease. This allows the patient to be notified of the imminent and/or end of the administration or discharge of the substance from the product container. Alternatively, the frequency of the perceptible discharge signal can be designed to be unchanging or constant.

The device according to the present disclosure can further include a switching sleeve arranged along the longitudinal axis (L) between the needle guard spring and the needle guard element, where the switching sleeve is carried along by the needle guard element in the proximal direction when the needle guard element is moving from the distal position to the proximal position. The switching sleeve, which is arranged between the needle guard spring and the needle guard element, particularly, may be moved from a distal position to a proximal position and back to the distal position, or to a distal position different from the distal position. The switching sleeve may be elongated and sleeve-shaped. The switching sleeve may be cylindrical, in particular circular-cylindrical.

The device according to the present disclosure can further include a locking sleeve arranged transversely to the longitudinal axis (L) between the switching sleeve and the holding element, where the locking sleeve in the distal position of the needle guard element prevents radial outward movement or radial deflection of the holding element from the engagement between the holding element and the injection spring. The locking sleeve is arranged to be axially displaceable from a distal position to a proximal position. The proximal end of the needle guard spring can be supported at the proximal end of the locking sleeve. Furthermore, the locking sleeve can be arranged to be axially movable on the holding element. The locking sleeve can be in a releasable engagement with the holding element in the distal position of the needle guard element. For example, the locking sleeve can have a projection, which can be releasably engaged in a recess on the holding element. Alternatively, the projection of the locking sleeve can engage around the distal end of the holding element, thus forming a releasable engagement.

In the proximal position of the needle guard element, the switching sleeve can release the locking sleeve and the holding element can move into the release position with the injection spring. The injection spring acts on the holding element in such a way that the holding element is moved radially outwardly out of engagement with the injection spring or is deflected radially. The locking sleeve disengages from the holding element. The force exerted by the needle guard spring thus allows the locking sleeve to be axially displaced or moved from the distal position into the proximal direction. The locking sleeve is movable in the proximal direction relative to the holding element fixed to the housing, and can come into stop contact with the holding element in order to generate an acoustic, visual and/or tactile discharge start signal. In this way, the patient can be notified when the administration or discharge of the substance from the product container has begun. The acoustic signal can for example be designed as an audible stop contact between the locking sleeve and the holding element. Furthermore, the housing of the device according to the present disclosure can for example have a window to visibly indicate the axial displacement of the locking sleeve relative to the holding element. During the axial movement between the locking sleeve and the holding element, a perceptible vibration can be generated in order to generate a tactile discharge signal. The locking sleeve may be elongated and sleeve-shaped. The locking sleeve may be cylindrical, in particular circular-cylindrical.

Furthermore, the locking sleeve and the switching sleeve can have a connecting piece guide, a locking stop and a corresponding tensionable locking element. The locking sleeve and the switching sleeve may be arranged to be axially movable relative to each other and rotatable relative to each other. The locking stop can be ramp-shaped, where the locking element can be deflected radially over the ramp of the locking stop during a movement in the distal direction and can snap into place behind a shoulder of the ramp-shaped locking stop. The locking element may be designed to be elastic. The locking element can be designed as an elastic locking arm or an elastic locking catch.

During the movement of the needle guard element in the proximal direction, the locking sleeve and the switching sleeve may be rotated relative to each other by the connecting piece guide between the locking sleeve and the switching sleeve, where the locking element can come into axial alignment with the locking stop in order to prevent a renewed or second movement of the needle guard element into the proximal position. The needle guard is thus locked and can protect the patient from being wounded by the needle or cannula, in particular by the distal tip of the needle or the distal tip of the cannula.

### BRIEF DESCRIPTION OF THE DRAWINGS

Implementations are described below with reference to a number of drawings. Both individually and in combination, the features disclosed herein advantageously develop the implementations further. In the drawings:

FIG. 1 shows a longitudinal sectional view of a first embodiment of the device according to the present disclosure with a longitudinal axis (L) in an as-delivered state.

FIG. 2 shows a longitudinal sectional view of the device according to FIG. 1 in an administered or discharged state.

FIG. 3 shows a longitudinal sectional view of the device according to FIG. 1 in a locked state.

FIG. 4 shows a longitudinal sectional view of an embodiment not according to the present disclosure of the device with a longitudinal axis (L) in an as-delivered state.

FIG. 5 shows a longitudinal sectional view of the device according to FIG. 4 in an administered or discharged state.

FIG. 6 shows a longitudinal sectional view of the device according to FIG. 4 in a locked state.

FIG. 7 shows a longitudinal sectional view of a second embodiment of the device according to the present disclosure with a longitudinal axis (L) in an as-delivered state.

FIG. 8 shows a longitudinal sectional view of the device according to FIG. 7 in an administered or discharged state.

FIG. 9 shows a longitudinal sectional view of the device according to FIG. 7 in a locked state.

### DETAILED DESCRIPTION

### Definitions

The term “medicament” here includes any flowable medical formulation suitable for controlled administration or discharge through a means such as a cannula or needle, for example comprising a liquid, a solution, a gel, or a fine suspension containing one or more active medical ingredients. A medicament can be a single active substance composition or a pre-mixed or co-formulated composition, having multiple active substances, from a single container. The term medicament includes drugs such as peptides (e.g. insulins, insulin-containing medicaments, GLP-1-containing and also derived or analogous preparations), proteins and hormones, biologically obtained or active substances, active substances based on hormones or genes, nutrient formulations, enzymes and further substances not only in solid (suspended) or liquid form but also polysaccharides, vaccines, DNS or RNS or oligonucleotides, antibodies or parts of antibodies, and suitable base, auxiliary and excipient substances.

In this document, the term “directly” means “directly” or “not separated from each other or only a short distance.” For example, in one embodiment of the device according to the present disclosure, no component (e.g. no piston rod) is arranged between the injection spring and the stopper. In one embodiment of the device according to the present disclosure, the injection spring can for example act directly on the stopper. Furthermore, for example, no component (e.g. a piston rod) is arranged between the injection spring and the product container, wherein there is only a short distance or no distance between the injection spring and the product container. The term “directly” can therefore be read not only as “along the longitudinal axis (L)” but also as “transversely to the longitudinal axis (L).”

In this document, the term “indirectly” means acting “indirectly” or acting “via an intermediate element.” For example, in another embodiment of the device, a component (e.g. an adapter) is arranged between the injection spring and the stopper. In another embodiment of the device, for example, the injection spring can act indirectly on the stopper. The term “indirectly” can be read as “along the longitudinal axis (L)” as well as “transversely to the longitudinal axis (L).”

### Detailed Description of the Figures

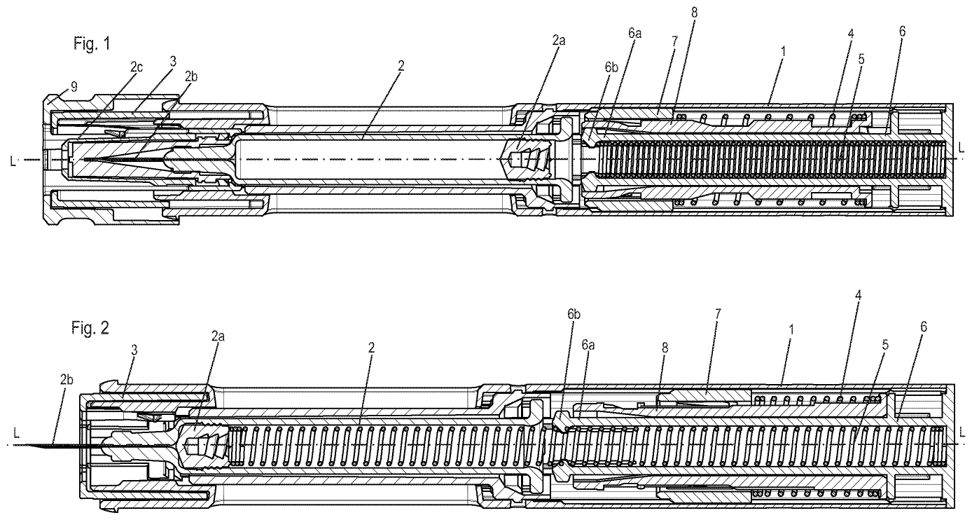

FIG. 1 shows a longitudinal sectional view of a first embodiment of the device according to the present disclosure in the form of an autoinjector with a longitudinal axis (L) in an as-delivered state. The device is used for administering or discharging a fluid substance, in particular a medicament. The device has a housing (1). The housing (1) can be sleeve-shaped. In the housing (1), a product container (2) with the substance to be administered is accommodated in an axially fixed manner. In the product container (2) a stopper (2a) is accommodated in axially displaceable fashion, in order to administer or discharge the substance from the product container (2). The device may include a needle (2b) through which the substance can be administered or discharged. The needle (2b) is provided at the distal end of the product container (2). Furthermore, the device may include a needle guard element (3). The needle guard element (3) serves to protect against puncture wounds from the needle (2b), in particular a distal tip of the needle (2b) of the product container (2). The needle guard element (3) can be sleeve-shaped. Furthermore, the device has a needle guard spring (4), which is coupled to the needle guard element (3), where the needle guard element (3) is axially movable from a distal position in which the distal tip of the needle (2b) is surrounded by the needle guard element (3) to a proximal position in which the distal tip of the needle (2b) is released from the needle guard element (3). The device may further include an injection spring (5), which provides the energy to effect the administration or discharge of the substance and which cooperates with the stopper (2a) in order to administer or discharge the substance from the product container through the needle (2b). In the as-delivered state of the device, the needle guard spring (4) and the injection spring (5) are provided as pre-tensioned pressure springs. The device also may include a holding element (6) arranged axially fixed relative to the housing (1), in which the injection spring (5) is received, in particular so as to be pre-tensioned and be relaxable. The holding element (6) is radially movable outwardly or is radially deflectable between an engagement position in which the holding element is engaged with the injection spring (5) and a release position in which the holding element (6) is disengaged from the injection spring (5). The holding element (6) may include a holding arm (6a). Furthermore, the holding arm (6a) may include a holding cam (6b). The holding arm (6a) and/or the holding cam (6b) is radially movable outwardly or radially deflectable. The holding arm (6a) of the holding element (6) extends from a proximal end to a distal end, where the proximal end of the holding element (6) or of the holding arm (6a) is axially fixed to the housing (1). The holding element (6) and/or the holding arm (6a) and/or the holding cam of the holding element (6) are designed to be elastic. The holding cam (6b) of the holding element (6) is designed such that the holding cam (6b) is radially movable outwardly or radially deflectable between the engagement position, in which the holding cam (6b) is engaged with the injection spring (5), in particular a coil or the coils of the injection spring (5), and a release position, in which the holding cam (6b) is disengaged from the injection spring (5), in particular from the coil or coils of the injection spring (5). In the as-delivered state of the device, the holding cam (6b) of the holding element (6) holds the injection spring (5) in pre-tensioned engagement. The device further may include a switching sleeve (7), which is arranged along the longitudinal axis (L) between the needle guard spring (5) and the needle guard element (3). The switching sleeve (7) can be sleeve-shaped. The switching sleeve (7) can be carried along in the proximal direction by the needle guard element (3) when the needle guard element (3) is moving from the distal position to the proximal position. The device further may include a locking sleeve (8), which is arranged transversely to the longitudinal axis (L) between the switching sleeve (7) and the holding element (6). The locking sleeve (8) can be sleeve-shaped. The locking sleeve (8) is arranged so as to be rotatable relative to the switching sleeve (7). The switching sleeve (7) is arranged in a rotationally fixed manner relative to the housing (1). In the distal position of the needle guard element (3), the locking sleeve (8) can prevent radial outward movement or radial deflection of the holding element (6), in particular of the holding cam (6b) of the holding element (6), out of the engagement between the holding element (6) and the injection spring (5). The proximal end of the needle guard spring (4) is supported on the proximal end of the locking sleeve (8). The locking sleeve (8) is arranged so as to be axially movable on the holding element (6). In the as-delivered state of the device, the locking sleeve (8) is in a releasable engagement with the holding element (6). The locking sleeve (8) can have a projection (not visible), which can be releasably engaged with a recess (not visible) in the holding element (6). In order for the patient to be able to use the device, in particular the autoinjector, for administering or discharging the substance, the patient must first remove a cap (9), which is detachably arranged on the device. Removing the cap (7) from the device causes the removal of a needle guard cap (2c), which is detachably attached to the needle (2a). The patient then presses the device against his skin, where the needle guard element (3) is moved from the distal position to the proximal position relative to the housing (1). In the process, the needle (2b) is inserted into the patient's skin. This relative movement of the needle guard element (3) with respect to the housing (1) causes the switching sleeve (7), acted upon by the needle guard spring (4), to be moved in the proximal direction relative to the housing (1). In the proximal position of the needle guard element (3), the switching sleeve (7) can release the locking sleeve (8). The holding cam (6b) of the holding element (6) can be radially movable outwardly or radially deflected due to the force of the injection spring (5), so that the holding cam (6b) of the holding element (6) moves out of engagement with the injection spring (5), in particular the coil or coils of the injection spring (5). The projection (not visible) of the locking sleeve (8) disengages from the recess (not visible) in the holding element (6). The projection (not visible) of the locking sleeve (8) moves radially outwards or is radially deflected and can form a connecting piece guide. The connecting piece guide of the projection (not visible) can engage with a connecting piece guide provided on the switching sleeve (7). The force acting on the needle guard spring (4) causes the locking sleeve (8) to move axially relative to the switching sleeve (7) and to the holding element (6) fixed to the housing in the proximal direction, and to come into stop contact with the holding element (6), where a discharge start signal is generated, in particular an audible stop. During the axial movement of the locking sleeve (8) in the proximal direction, the locking sleeve (8) rotates relative to the switching sleeve (7) due to the connecting piece guide between the projection (not visible) of the locking sleeve (8) and the switching sleeve (7). Due to the relative movement between the locking sleeve (8) and the switching sleeve (8), a locking stop (not visible) provided on the locking sleeve (8) comes into axial alignment with a locking element (not visible) provided on the switching sleeve (7). In an alternative embodiment of the device according to the present disclosure, the locking sleeve can have the locking element and the switching sleeve can have the locking stop.

In the first embodiment of the device according to the present disclosure, the injection spring (5) acts directly on the stopper (2a) of the product container (2). Furthermore, the injection spring (5) relaxes directly into the product container (2) during the administration or discharge of the fluid substance. As a result, a device can be provided that has a high administration or discharge rate and a compact design. During the administration or discharge of the fluid substance from the product container (2), the coils of the injection spring (5) relax relative to the holding element (6) and over the holding cam (6b) of the holding arm (6a) in order to generate a discharge signal, in particular an acoustic discharge signal in the form of an audible rattling or clicking. The holding element (6) and/or the holding arm (6a) and/or the second holding cam (6b) may be configured as a click element. At the end of administration or discharge, as shown in FIG. 2, in particular the acoustic discharge signal ceases, indicating to the patient that the administration or discharge of the fluid substance from the product container (2) has ended and the patient can remove the device from his skin. When the device is removed from the skin, due to the force of the needle guard spring (4), the needle guard element (3) and the switching sleeve (7) move from the proximal position back to the distal position, or to another distal position different from the distal position.

The switching sleeve (7) may include the locking element (not visible), which may be designed to be elastic. The locking element (not visible) may be radially deflectable. The locking sleeve (8) may include the locking stop (not visible). Due to the axial and rotational relative movement between the locking sleeve (8) and the switching sleeve (7), the locking stop (not visible) of the locking sleeve (8) may come into axial alignment with the locking element (not visible) of the switching sleeve (7). The locking stop (not visible) of the locking sleeve (8) may be ramp-shaped with a shoulder (not visible), where the shoulder (not visible) can be arranged at a distal end of the locking stop (not visible).

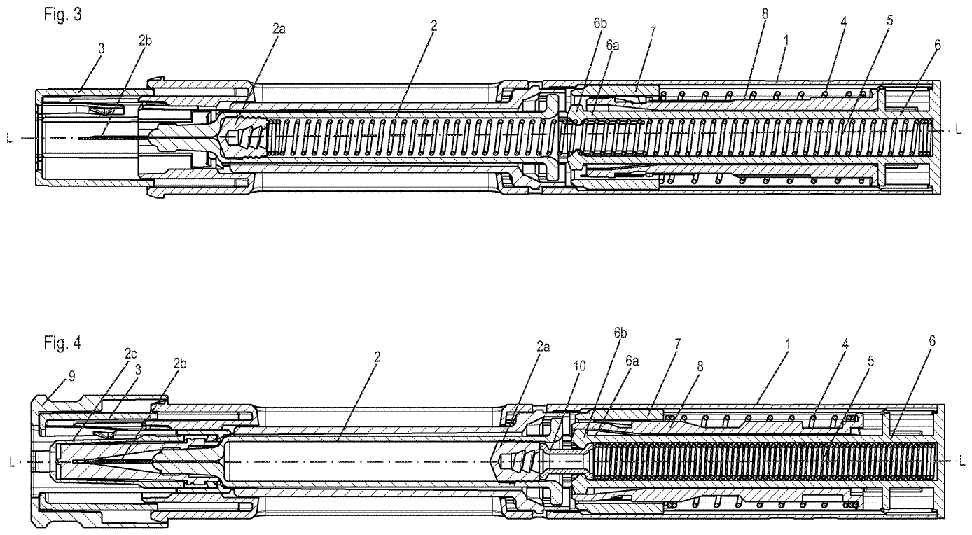

During the movement of the needle guard element (3) and switching sleeve (7) from the proximal position to the distal position or to a distal position different from the distal position, where the switching sleeve (7) and the needle guard element (3) are moved in the distal direction by the force of the needle guard spring (4), the locking element (not visible) of the switching sleeve (7) slides over the ramp-shaped locking stop (not visible) of the locking sleeve (8). The locking element (not visible) of the switching sleeve (7) can relax and/or snap into place behind the shoulder (not visible) of the locking stop (not visible) of the locking sleeve (8). When there is a renewed or second movement of the needle guard element (3) in the proximal direction, the locking element (not visible), in particular the relaxed locking element (not visible) of the switching sleeve (7), can come into stop contact with the locking stop (not visible), in particular with the shoulder (not visible) of the locking element (not visible) of the locking sleeve (8). As a result, the needle guard element (3) may be locked and can protect the patient from being wounded by the needle (2), in particular by the distal tip of the needle (2b). The device is in a locked state, as shown in FIG. 3. Alternatively, the locking stop can be provided on the switching sleeve (7) and the locking element can be provided on the locking sleeve (8).

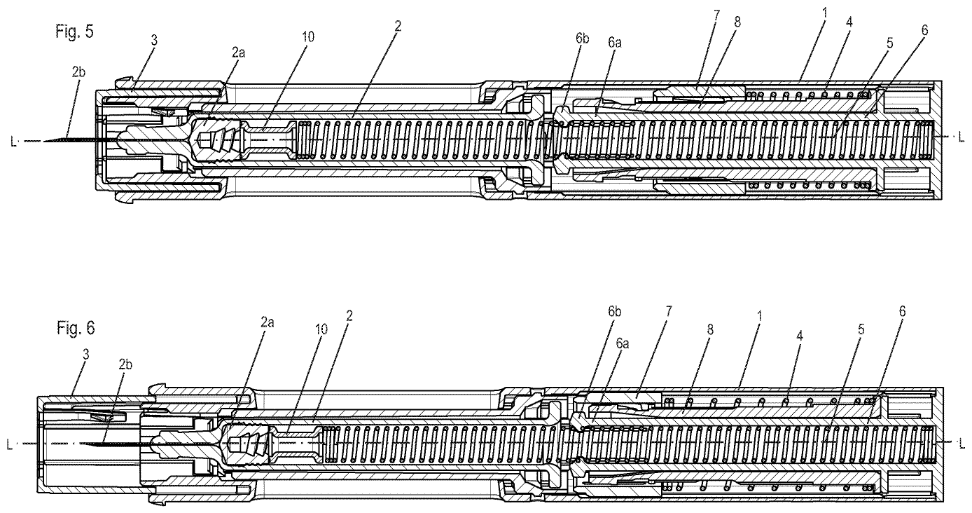

FIGS. 4, 5 and 6 show an embodiment not according to the present disclosure of the device for administering or discharging a fluid substance, where the device is shown in an as-delivered state in FIG. 4, in an administered or discharged state in FIG. 5, and in a locked state in FIG. 6. This embodiment differs from the first embodiment of the device by the presence of an additional adapter (10). Otherwise, the design and function of this embodiment correspond to the first embodiment of the device, previously described. The adapter (10) is arranged between the injection spring (5) and the stopper (2a) of the product container (2). The adapter (10) is arranged axially between the injection spring (5) and the stopper (2a) of the product container (2). The injection spring (5) thus acts via the adapter (10) on the stopper (2a) in order to administer or discharge the substance. The engagement element (6) may include a holding arm (6a) and a holding cam (6b). The engagement element (6), in particular the holding arm (6a) and/or the holding cam (6b), may be elastic. The holding cam (6b) can be engaged with the adapter in an as-delivered state of the device and can be disengaged from the adapter in order to administer or discharge the substance. The holding element (6) is radially movable outwardly or is radially deflectable between an engagement position in which the holding element is engaged with the adapter (10) and a release position in which the holding element (6) is disengaged from the adapter (10). The adapter (10) can be sleeve-shaped. In this way, differently shaped injection springs (5) and differently shaped stoppers (2a) can be assembled. Furthermore, the adapter (10) may be configured as a damping element in order to reduce or correspondingly adjust the force of the injection spring (5). The injection spring (5) acts indirectly on the stopper (2a) of the product container (2). Furthermore, the injection spring (5) relaxes directly into the product container (2) during the administration or discharge of the fluid substance. As a result, a device can be provided that has a high administration or discharge rate and a compact design.

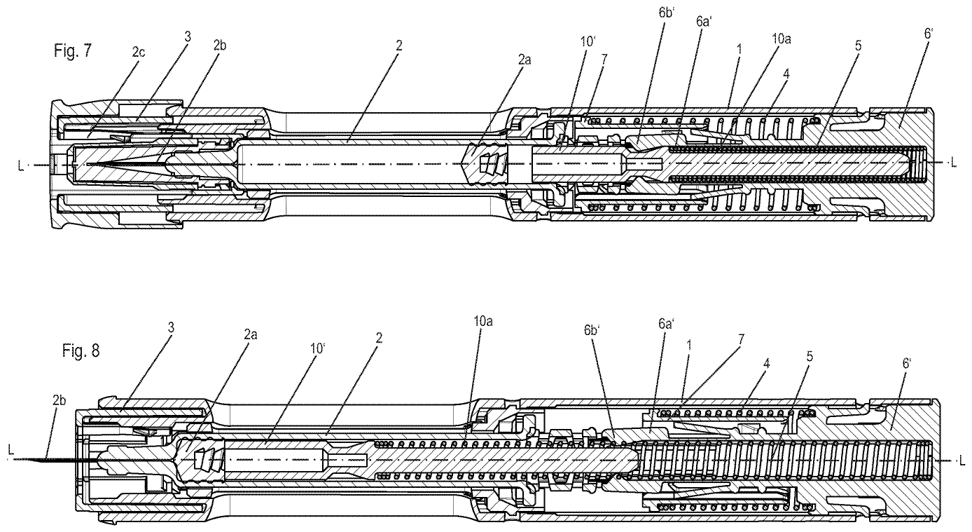

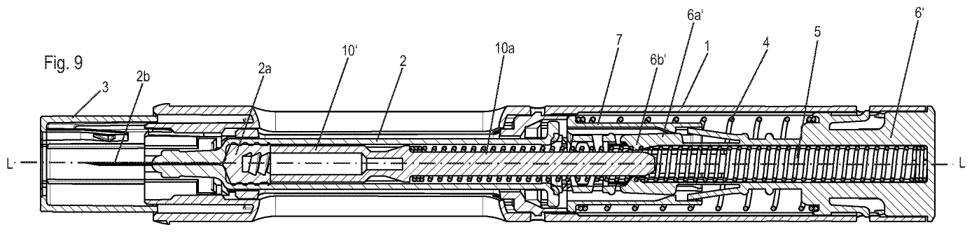

FIGS. 7, 8 and 9 show a second embodiment of the device according to the present disclosure for administering or discharging a fluid substance, wherein the device is shown in an as-delivered state in FIG. 7, in an administered or discharged state in FIG. 8, and in a locked state in FIG. 9. The second embodiment differs from the first embodiment of the device according to the present disclosure by the shape of the holding element (6′) and by the presence of an additional adapter (10′). The holding element (6′) has a first holding arm (not visible) with a first holding cam (not visible), which is radially movable outwards or radially deflectable from an engagement position in which the holding cam (not visible) is engaged with the coil or coils of the injection spring (5) into a release position in which the holding cam (not visible) is disengaged from the coil or coils of the injection spring (5). The holding cam (not visible) can be engaged with the injection spring in the as-delivered state and can be disengaged from the injection spring in order to administer or discharge the substance. The holding element (6′) also has a second holding arm (6a′) with a second holding cam (6b′). The second holding arm (6a′) is elongated and extends along the longitudinal axis (L). Alternatively, a second holding element, which is axially fixed to the housing or is a part fixed to the housing, can also be provided. This second holding element can include the second holding arm (6a′) and the second holding cam (6b′). The second holding arm (6a′) and the second holding cam (6b′) are arranged radially offset by 90 degrees to the first holding arm (not visible) and to the first holding cam (not visible). The second holding arm (6a′), in particular the second holding cam (6b′), is designed in such a way as to generate a release signal, in particular an acoustic release signal in the form of an audible rattling or clicking, during the administration or discharge of the substance. When the injection spring (5) is relaxed, the holding cam (6b′) slides over the coils of the injection spring (5). During the administration or discharge of the fluid substance from the product container (2), the coils of the injection spring (5) relax relative to the holding element (6) and over the holding cam (6b′) of the holding arm (6a′) to generate a discharge signal, in particular an acoustic discharge signal in the form of an audible rattling or clicking. At the end of administration or discharge, as shown in FIG. 8, in particular the acoustic discharge signal ceases, indicating to the patient that the administration or discharge of the fluid substance from the product container (2) has ended. The holding element (6′) and/or the second holding arm (6a′) and/or the second holding cam (6b′) may be configured as a click element. The holding element (6′) and/or the second holding arm (6a′) and/or the second holding cam (6b′) may be elastic. A plurality of first (not visible) and/or second holding arms (6a′) with the corresponding holding cams (6b′) can be provided on the holding element (6′). The distal end of the injection spring is supported at a distal end of the adapter (10′) and the proximal end of the injection spring is supported at a proximal end of the holding element (6′). The second holding cam (6b′) can be engaged with the adapter (10′) in an as-delivered state of the device and can be disengaged from the adapter (10′) in order to administer or discharge the substance. The holding cam (6b′) is radially movable outwards or radially deflectable between an engagement position in which the holding cam (6b′) is engaged with the adapter (10′) and a release position in which the second holding cam (6b′) is disengaged from the adapter (10′). The adapter (10′) can be sleeve-shaped. In this way, differently shaped injection springs (5) and differently shaped stoppers (2a) can be assembled. Furthermore, the adapter (10′) may be configured as a damping element to reduce or correspondingly adjust the force of the injection spring (5). The injection spring (5) acts indirectly on the stopper (2a) of the product container (2). Furthermore, the injection spring (5) relaxes directly into the product container (2) during the administration or discharge of the fluid substance. As a result, a device can be provided that has a high administration or discharge rate and a compact design. The adapter (10′) has a guide (10a). The guide (10′) is arranged on an outer surface of the adapter (10′) so that the injection spring (5) is guided when the injection spring (5) is relaxed. The guide (10a) extends parallel to the spring axis of the injection spring (5). This can prevent the injection spring from bending.

In a further embodiment of the device according to the present disclosure, an additional sleeve (not visible) that is movable relative to the housing can be provided, which is movable over the second holding arm (6a′) with the second holding cam (6b′) in order to generate a discharge signal, in particular an acoustic discharge signal in the form of an audible rattling or clicking. This sleeve (not visible) can be arranged concentrically to the longitudinal axis (L) between the injection spring (5) and the second holding cam (6b″). This sleeve (not visible) can have one or more recesses and/or one or more projections which slide or ratchet successively over the second holding cam (6b″) in order to generate the discharge signal. These multiple recesses are arranged along the longitudinal axis (L). These multiple recesses can vary in size. If multiple second holding arms (6a′) and multiple second holding cams (6b′) are provided, corresponding recesses can be provided along the longitudinal axis (L) which interact with these second holding cams (6b′) in order to generate a discharge signal. These corresponding recesses can be arranged offset from each other along the longitudinal axis (L) or at the same axial position along the longitudinal axis (L).

### REFERENCE SIGNS

1 Housing
2 Product container
2a Stopper
2b Needle
2c Needle guard cap
3 Needle guard element
4 Needle guard spring
5 Injection spring
6, 6′ Holding element
6a Holding arm
6a′ Second holding arm
6b Holding cam
6b′ Second holding cam
7 Switching sleeve
8 Locking sleeve
9 Cap
10, 10′ Adapter
10a Guide
L Longitudinal axis

What is claimed is:

1. A device for administering or discharging a fluid substance having a longitudinal axis, comprising:
a housing;
a product container axially fixed within the housing, the product container containing the substance to be administered and comprises a stopper axially displaceable therein and a needle provided thereon for administering or discharging the substance;
a needle guard element configured to be movable relative to the housing for protecting the needle;
a needle guard spring coupled to the needle guard element, wherein the needle guard element is configured to be axially movable from a distal position in which a distal tip of the needle is surrounded by the needle guard element to a proximal position in which the distal tip of the needle is released from the needle guard element;
an injection spring configured to provide energy to effect administration of the substance and to cooperate with the stopper to administer or discharge the substance from the product container through the needle; and
a holding element axially fixed relative to the housing, wherein the injection spring is accommodated in the holding element,
wherein the holding element is radially movable outwards or is radially deflectable between an engagement position in which the holding element is engaged with the injection spring and a release position in which the holding element is disengaged from the injection spring, and
wherein, during an axial movement of the needle guard element from the distal position to the proximal position, the holding element disengages from the injection spring such that the substance is administered or discharged from the product container through the needle.

2. The device according to claim 1, wherein the injection spring acts directly on the stopper to administer or discharge the substance.

3. The device according to claim 1, wherein the injection spring acts on the stopper via an adapter arranged along the longitudinal axis between the injection spring and the stopper to administer or discharge the substance.

4. The device according to claim 1, wherein the needle guard spring, the injection spring, or both are configured as a helical spring.

5. The device according to claim 4, wherein, when administering or discharging the substance, coils of the injection spring are relaxed relative to and over the holding element such that the coils thereby generate an acoustic, visual and/or tactile dispense signal.

6. The device according to claim 1, further comprising a switching sleeve arranged along the longitudinal axis between the needle guard spring and the needle guard element, wherein when the needle guard element moves from the distal position to the proximal position, the switching sleeve is slaved by the needle guard element in a proximal direction.

7. The device according to claim 6, further comprising a locking sleeve arranged transversely to the longitudinal axis between the switching sleeve and the holding element, wherein in the distal position of the needle guard element, the locking sleeve prevents a radial outward movement or radial deflection of the holding element from the engagement between the holding element with the injection spring.

8. The device according to claim 7, wherein the switching sleeve, in the proximal position of the needle guard element, releases the locking sleeve, and the holding element moves into the release position with the injection spring.

9. The device according to claim 7, wherein the locking sleeve and the switching sleeve are arranged to be axially movable and rotatable relative to each other, and wherein after the movement of the needle guard element from the distal position to the proximal position, the locking sleeve and the switching sleeve are configured come into stop contact to prevent a second movement of the needle guard element into the proximal position.

10. The device according to claim 7, wherein the locking sleeve is movable in the proximal direction relative to the holding element into stop contact with the holding element to thereby generate an acoustic, visual, and/or tactile discharge start signal.

11. A device for dispensing a fluid substance, comprising:
a housing defining a longitudinal axis;
a product container axially fixed in the housing, the product container comprising a stopper axially displaceable therein to dispense the fluid substance through a needle provided on the product container,
an injection spring which, when expanding, cooperates with the stopper to dispense the fluid substance;
a holding element arranged axially fixed relative to the housing, in which the injection spring is accommodated and which holding element is radially deflectable between an engagement position in which the holding element is engaged with the injection spring and a release position in which the holding element is disengaged from the injection spring and the injection spring is enabled to expand;
a needle guard element, biased by a needle guard spring and axially movable from a distal position in which a distal tip of the needle is surrounded by the needle guard element to a proximal position in which the distal tip of the needle is released from the needle guard element, to trigger or enable a radial deflection of the holding element into the release position.

12. The device according to claim 11, wherein the injection spring acts directly on the stopper to dispense the liquid substance.

13. The device according to claim 11, wherein the injection spring acts on the stopper via an adapter which is arranged along the longitudinal axis between the injection spring and the stopper to dispense the liquid substance.

14. The device according to claim 11, wherein the needle guard spring, the injection spring, or both are configured as a helical spring.

15. The device according to claim 14, wherein, when dispensing the liquid substance, coils of the injection spring are relaxed relative to and over the holding element such that the coils thereby generate an acoustic, visual and/or tactile dispense signal.

16. The device according to claim 11, further comprising a switching sleeve arranged along the longitudinal axis between the needle guard spring and the needle guard element, wherein when the needle guard element moves from the distal position to the proximal position, the switching sleeve is slaved by the needle guard element in a proximal direction.

17. The device according to claim 16, further comprising a locking sleeve arranged between the switching sleeve and the holding element, wherein, in the distal position of the needle guard element, the locking sleeve prevents the radial deflection of the holding element from the engagement between the holding element with the injection spring.

18. The device according to claim 17, wherein the switching sleeve, in the proximal position of the needle guard element, releases the locking sleeve, and the holding element moves into the release position with the injection spring.

19. The device according to claim 17, wherein the locking sleeve and the switching sleeve are arranged to be axially movable and rotatable relative to each other, and wherein after the movement of the needle guard element from the distal position to the proximal position the locking sleeve and the switching sleeve are configured to come into stop contact to prevent a second movement of the needle guard element into the proximal position.

20. The device according to claim 17, wherein the locking sleeve is movable in the proximal direction relative to the holding element into stop contact with the holding element to thereby generate an acoustic, visual, and/or tactile discharge start signal.
